(19) United States
(12) Patent Application Publication
(10) Pub. No.: US 2014/0335583 A1
(43) Pub. Date: Nov. 13, 2014

(54) Methods and Systems for Producing Products Using Engineered Iron Oxidizing Bacteria

(71) Applicants: Alan West, Tenafly, NJ (US); Scott Banta, Fairfield, CT (US)

(72) Inventors: Alan West, Tenafly, NJ (US); Scott Banta, Fairfield, CT (US)

(21) Appl. No.: 14/444,527

(22) Filed: Jul. 28, 2014

Related U.S. Application Data
(63) Continuation-in-part of application No. 13/823,419, filed on Sep. 20, 2013.
(60) Provisional application No. 62/028,043, filed on Jul. 23, 2014.
(60) Provisional application No. 61/858,998, filed on Jul. 26, 2013.

(51) Int. Cl. C12P 7/16; C12P 5/02; C12P 7/04

(52) U.S. Cl. CPC C12P 7/16; C12P 7/04; C12P 5/02
USPC 435/160; 435/294.1; 435/157; 435/167

(57) ABSTRACT

Methods and systems for producing a biofuel using genetically modified iron-oxidizing bacteria (IOB) are disclosed. In some embodiments, the methods include the following: providing an IOB that have been genetically modified to enable them to generate a biofuel or chemical; feeding a first source of ferrous iron to the IOB; feeding water, carbon dioxide, and oxygen to the IOB; producing at least the biofuel or chemical, ferric iron, and an IOB biomass; and preventing ferric precipitates from forming. In some embodiments, the methods and systems include the following: a bioreactor including IOB that have been genetically modified to enable them to generate a biofuel; a first source of ferrous iron; sources of water, carbon dioxide, and oxygen; a source of anti-ferric precipitating agent in fluid communication with the bioreactor; and a electrochemical reactor that is configured to electrochemically reduce ferric iron to a second source of ferrous iron.

### CROSS REFERENCE TO RELATED APPLICATION(S)

This application claims the benefit of U.S. Provisional Application Nos. 61/858998, filed Jul. 26, 2013, and 62/028043, filed Jul. 23, 2014, and is a continuation-in-part of U.S. application Ser. No. 13/823419, which was the National Stage of International Application No. PCT/US2012/026697, filed Feb. 27, 2012, each of which is incorporated by reference as if disclosed herein in its entirety.

### BACKGROUND

There has been interest in the development of liquid biofuels as these processes have the potential to directly fix carbon dioxide into transportation fuels, which is potentially carbon neutral and politically attractive. Cellulose based biofuels including bioethanol, algae-derived lipids, cyanobacteria, and algae derived hydrogen (H₂) are among the most studied biofuels. Despite the promise of these technologies and processes, there are specific limitations that preclude their wide-spread application. For example, post-processing of algal cells and derived lipids imposes higher production costs on algal biodiesel. The production rates of H₂ from cyanobacteria still remains low and productivity needs to be improved. Genetically engineered photosynthetic organisms have also been explored for bioethanol production. However separation of ethanol from the aqueous phase remains a challenge.

Microbial fuel cells have been under investigation and development for more than a century, as the use of cells to harvest electrical energy from waste streams is attractive for many reasons. In a biofuel cell, biological catalysts are used on an anode to oxidize biofuels, and a cathode is created that can use the generated electrons to reduce oxygen to water. These systems can either be microbial with living cells on the electrodes, or they can be enzymatic systems, with purified enzymes on the electrodes. In both designs, power can be generated from the oxidation of biofuels, and there are many advantages to these systems over conventional fuel cells and other power generation schemes. However, much research still needs to be done with microbial fuel cells to make them practical and cost-efficient. A significant limitation for both enzymatic and microbial fuel cells is the need for mediators to enable electrical contact between the biological components and inorganic electrode. In some microbial systems, these mediators are made by the organisms themselves, and in other technologies, synthetic mediators are added to the system. In some systems, cells must make physical contact with the electrodes for electron transfer. This can be a significant limitation as it reduces the cellular mass that can be used for biochemical conversion.

There is an intense global interest in the development of next-generation biofuels. Currently, biofuels are produced using the metabolic activities of heterotrophic organisms that transform organic materials, such as sugar, into fuels such as ethanol. Autotrophic bacteria have recently attracted attention as potential biosynthetic platforms for chemical production since autotrophs do not require organic compounds as substrates and thus do not involve agriculture. More recently, there has been interest in producing fuels and chemicals using electricity (termed electrofuels) and autotrophic bacteria such as Acidithiobacillus ferrooxidans (“A. ferrooxidans”), Nitrosomona europaea, Sporomusa ovata, and Ralstonia eutropha. A. ferrooxidans cells are an attractive candidate for this approach as they grow planktonically and the inorganic Fe³⁺ produced by the cells are readily reduced electrochemically. Thus, soluble iron is recycled between an electrochemical reactor and a bioreactor containing genetically engineered A. ferrooxidans cells. The cells grow continuously using electrochemically reduced iron while fixing gaseous CO₂. In these types of processes, it is desired that the oxidized iron is kept soluble at high concentrations.

A. ferrooxidans cells grow optimally at temperatures ranging from 28 degrees Celsius to 33 degrees Celsius and at a pH range from 2.0 to 2.3. In laboratory cultures, the cells extract energy from the oxidation of Fe²⁺ to Fe³⁺ through the following process:

4FeSO₄+2H₂SO₄+O₂=2Fe₂(SO₄)₃+2H₂O (1)

Under these conditions, the solubility of Fe³⁺ product is very low: at pH 2.0, less than 40 mM of Fe³⁺ is soluble. This can be advantageous, as Fe³⁺ has been shown to be a competitive inhibitor for the growth of A. ferrooxidans and thus precipitation of ferric iron lowers the extent of inhibition. In the most widely used laboratory media recipes, such as 9-K medium, more than 40 mM Fe2+ is included to ensure high cell yield and under these conditions, the oxidized iron precipitates. Unfortunately, the precipitation of iron complicates the electrochemical reduction of Fe³⁺ in an iron recycling process. Precipitation can be prevented by operating at lower pH values or by using lower concentrations of Fe²⁺ in the medium.

Growth at low pH can be used to address precipitation, but this reduces the cellular growth rate and yield. This is due to the fact that A. ferrooxidans cells maintain a circumneutral cytoplasmic pH at around 6.5. The cells have evolved several mechanisms to maintain this proton gradient across the membrane and some of the pH homeostasis mechanisms are ATP-dependent. Cells both promote proton outflow using active proton pumps and reduce proton inflow through a Donnan potential generated by active transport of potassium ions. Therefore it is logical to assume that while growth at low pH addresses issues with iron precipitation, it will likely increase maintenance coefficient (the amount of energy required for cell maintenance at zero growth), enhance product inhibition by soluble Fe³⁺ and reduce cell yield.

For an iron system to reach high concentrations, it must be at very low pH values. However, ferric ions have solubility limitations due to the formation of insoluble precipitates at pH values above 2.0. For a total iron concentration of 70 mM, nearly all of it will precipitate to ferric hydroxide by pH 2.5, limiting the ranges of feasible working pH values. This solubility limit must either be overcome or circumvented by other means if the necessary working conditions are to be achieved.

### SUMMARY

Living cells have the ability to reproduce and maintain their catalytic machinery, and their metabolic pathways can be rationally altered to meet desired process objectives. But, efficient electron transfer from the electrode to the organism can limit metabolic production, and the use of mediating species can result in a process that is not economically viable. One way to address these limitations is to explore alternative organisms that naturally utilize mediators that are more attractive. The disclosed subject matter includes the metabolic engineering of chemolithoautotrophic iron-oxidizing-bacteria (IOB), such as A. ferrooxidans, to develop a process that can overcome these limitations. IOB have the natural ability to fix carbon dioxide while oxidizing ferrous iron (Fe²⁺) to ferric iron (Fe³⁺).

Referring to FIGS. 1 and 2, aspects of the disclosed subject matter include the use of engineered strains of the IOB, e.g., A. ferrooxidans, for the production of biofuels. IOB fix carbon dioxide for cell-synthesis while deriving energy from the oxidation of ferrous iron to ferric iron. The ferric iron produced upon ferrous iron oxidation can be electrochemically reduced back to ferrous iron in an electrochemical reactor, and additional ferrous iron can be added from any ferrous iron-rich stream. In this way, the IOB can be grown efficiently in a bioreactor using ferrous iron as the mediator.

In methods and systems according to the disclosed subject matter, the precipitation of ferric ions in the bioreactor, which typically occurs at pH values above 2.0, was prevented by either adding one or more iron chelators to the fluid media to improve solubility of the iron or mixing the ferrous iron with additional metallic ions having a higher solubility, e.g., vanadium, to provide the necessary energy input while having a lower concentration of iron.

### BRIEF DESCRIPTION OF THE DRAWINGS

The drawings show embodiments of the disclosed subject matter for the purpose of illustrating the invention. However, it should be understood that the present application is not limited to the precise arrangements and instrumentalities shown in the drawings, wherein:

FIG. 1 is a schematic diagram of methods and systems according to some embodiments of the disclosed subject matter;

FIG. 2 is a schematic diagram of methods and systems according to some embodiments of the disclosed subject matter;

FIG. 3 is a chart of a method according to some embodiments of the disclosed subject matter; and

FIG. 4 is a chart of a method according to some embodiments of the disclosed subject matter.

### DETAILED DESCRIPTION

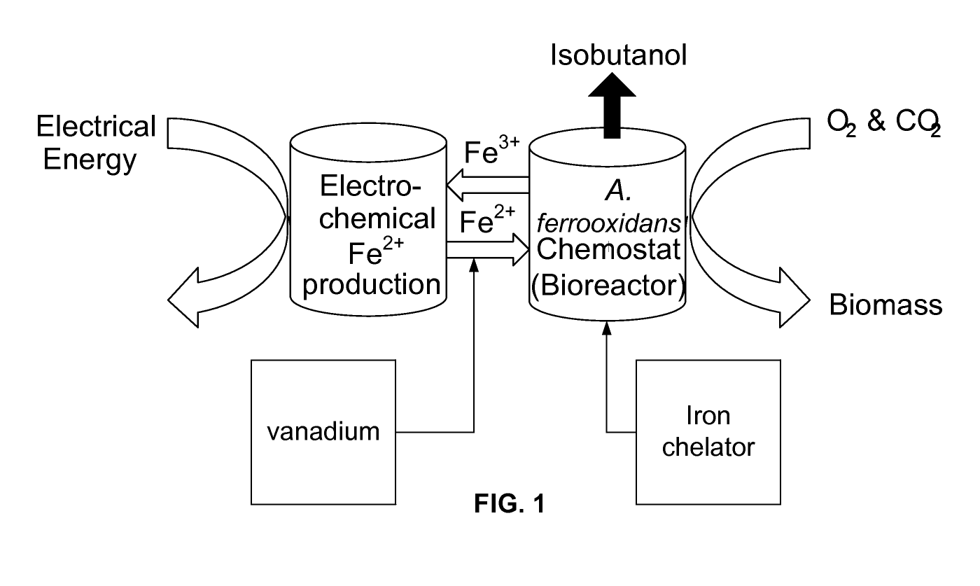

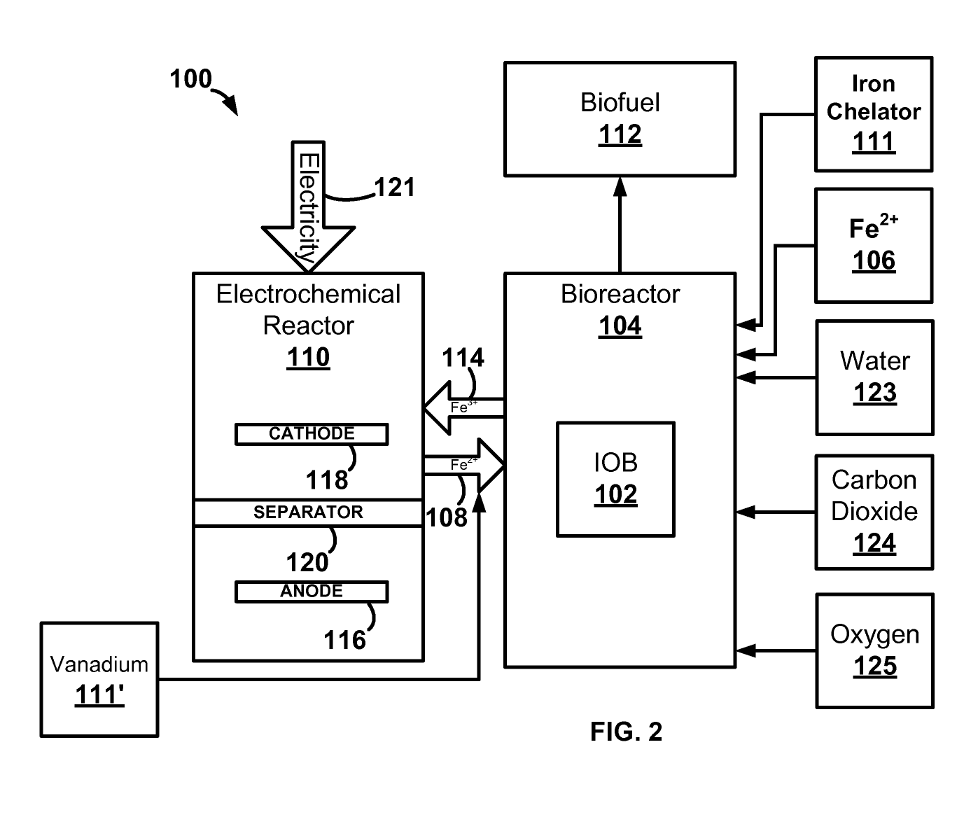

Referring again to FIGS. 1 and 2, aspects of the disclosed subject matter include methods and systems that include the application of chemolithoautotrophic IOB for concomitant carbon dioxide fixation, conversion of the carbon dioxide to a biofuel such as isobutanol, and oxidation of ferrous iron to ferric iron. The ferric iron produced upon ferrous iron oxidation is electrochemically reduced back to ferrous iron. Additional ferrous iron can be added from other ferrous iron-rich sources. Metabolic engineering is used to introduce a new pathway into the bacteria that starts with the precursors for amino acid synthesis to create butanols, e.g., isobutanol, etc. In order to eliminate ferric precipitates, the IOB are either mixed with one or more iron chelators or the ferrous iron is maintained at lower composition by mixing with an additional soluble redox mediator such as vanadium (V4+/V3+ couple).

Referring now to FIGS. 2-4, some embodiments include systems and methods for producing products such as biofuels and chemicals. As shown in FIG. 2, some embodiments include a system 100 for producing biofuels using genetically modified IOB 102 grown in a bioreactor 104 that are fed ferrous iron and carbon dioxide. The ferrous iron provides electrons to the IOB and the carbon dioxide is used as a base material to be fixed into a biofuel or chemical. Initially, the ferrous iron is typically provided from a first source 106 that is external to system 100, e.g., a ferrous iron-rich stream in fluid communication with bioreactor 104. Typically, but not always, the ferrous iron used in system 100 is substantially provided by a second source 108 that is generated by an electrochemical reactor 110. Second source 108 of ferrous iron serves as a mediator for transferring electrons to IOB 102. In some embodiments, substantially all of the ferrous iron used by bioreactor 104 is provided by a source external to system 100. System 100 also includes at least one source of an anti-ferric precipitating agent 111, 111′, for preventing ferric ions from precipitating in bioreactor 104.

Bioreactor 104 includes IOB 102 that have been genetically modified to include a particular metabolic pathway to enable them to generate a particular biofuel 112. The operating parameters of bioreactor 104 are typically optimized to maximize the production of ferric iron 114. In some embodiments, bioreactor 104 will be configured so as to be fed 70 mM ferrous iron. In others embodiments, bioreactor 104 will be configured so as to be fed as much as 500 mM ferrous iron. In some embodiments, the pH will likely be maintained in the range of about 1.5 to 4 and temperature at about 20 to 40 degrees Celsius. Methods and systems according to the disclosed subject matter have operating conditions that are optimized for optimal yield, conversion, etc. Bioreactors included in methods and systems according to the disclosed subject matter are typically operated in a continuous flow mode to maximize the conversion of the substrates to the products.

Ferric iron 114, which is generated in bioreactor 104, is introduced to electrochemical reactor 110, which is in fluid communication with the bioreactor. Electrochemical reactor 110 includes electrodes, i.e., an anode 116 and a cathode 118, a separator 120, and source of electrical energy 121. In some embodiments, cathode 118 is formed substantially from nickel or glassy carbon and anode 116 is formed from materials known in the art. In some embodiments, flow through or flow by porous electrodes are used. In some embodiments, separator 120 is a cation selective membrane, to allow for proton transfer across the membrane to achieve acid balance.

Electrochemical reactor 110 is typically configured to electrochemically reduce ferric iron 114 to second source 108 of ferrous iron using source of electrical energy 121. In system 100, ferric iron 114 will be continually regenerated back to ferrous iron, i.e., second source 108, and the recycle loop can be theoretically closed without the need for additional ferrous iron input from first source 106 beyond startup.

In some embodiments, a portion of the ferrous iron provided to bioreactor 104 is obtained from a ferrous iron-rich stream and a portion is obtained from electrochemical reactor 110.

Some embodiments of the disclosed subject matter include systems having holding tanks for the ferrous iron rich streams and ferric iron rich streams to enable the electrochemical production of ferrous iron to operate independently of the bioreactor to take advantage of the transient pricing and availability of electricity. For example, at times during the day when electricity is least expensive, the electrochemical system would produce as much ferrous iron as possible to be stored and used slowly by the bioreactor, which will be operating continuously. This solves a major limitation encountered in photobioreactors where interruptions in light can negatively impact the process.

System 100 includes a source of water 123, a source 124 of carbon dioxide, and a source of oxygen 125, all of which are in fluid communication with bioreactor 104. In some embodiments, source 124 is carbon dioxide removed from air or energy plant emissions. In some embodiments, either in place of or in addition to carbon dioxide, carbonate, e.g., from mineral sources, is fed to bioreactor 104.

System 100 includes one or more sources of anti-ferric precipitating agents 111, 111′, for preventing ferric ions from precipitating in bioreactor 104. In some embodiments, the anti-ferric precipitating agents include an iron chelator 111, e.g., one or more of malonic acid, citric acid, gluconic acid, or similar. In some embodiments, iron chelator 111 includes citric acid at a concentration of about 70 mM. Addition of citric acid to the medium eliminated ferric precipitation even at concentrations up to 280 mM, allowing for the growth of A. ferrooxidans at higher pH and larger Fe²⁺ concentrations.

In some embodiments, the anti-ferric precipitating agents include an indirect electron supplier 111′, e.g., vanadium or a similar metallic ion. In some embodiments, the vanadium (V³⁺) has a concentration of about 60 mM and the ferrous iron has a concentration of about 10 mM. In some embodiments that utilize an indirect electron supplier 111′, in order to provide a medium with high ionic conductivity, salt is added. In some embodiments, Mg²⁺ was added due to its less toxic effect on the bacterium. In some embodiments, Mg²⁺ having a concentration of 500 mM was used considering constraints on bacterial growth and electrochemistry.

The energy density of the media is proportional to Fe²⁺ concentration, but the soluble Fe³⁺ concentration is significantly limited by pH. Theoretically at a pH of 2.2 less than 10 mM Fe³⁺ is soluble. On the other hand, a high concentration of Fe²⁺ or Fe³⁺ may also impede cell growth due to substrate or product inhibition. Fe²⁺ concentration higher than 45 mM¹³ and Fe³⁺ concentration higher than 36 mM¹⁴ have been reported to inhibit cell growth. The use of vanadium as an indirect energy supplier is able to address the two problems. A. ferrooxidans has already been employed to extract vanadium from spent catalysts, where sulfur species of high reducing power were exploited. Here redox reactions between vanadium and iron ions are used. At a pH of 2.2, the culture growing in media of 10 mM Fe²⁺ supplemented with 60 mM V³⁺ achieved similar growth rate as an all iron culture. By using V³⁺ augmented with a small amount of Fe²⁺, both the solubility limitation and ferric inhibition are resolved. At the same time, the mixed media contains similar energy density. This indicates that by using vanadium, it is possible to improve the energy density of the media while not causing precipitation and inhibition.

Although not included in FIG. 2, in some embodiments, system 100 includes pumps for pumping the various constituents, into, through, and out of the system. In addition, the pumps are typically programmable to allow electrochemical reactor 110 to be turned off when the price of electricity is high and turned on when the price is low. Also, the pumps typically include a separator unit to separate one or more particular constituents that are to be pumped to other components of system 100 from the other constituents.

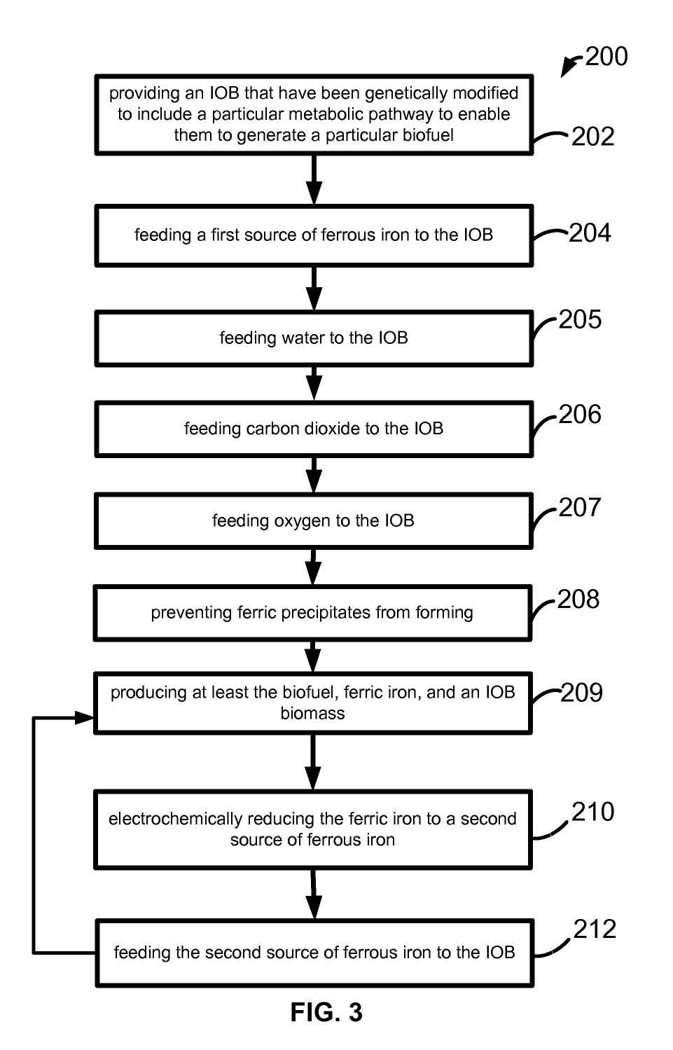

Referring now to FIGS. 2-4, some embodiments include a method 200 for producing a biofuel using genetically modified IOB. As shown in FIG. 3, at 202, IOB that have been genetically modified to include a particular metabolic pathway to enable them to generate a particular biofuel are provided.

In some embodiments, the IOB is substantially A. ferrooxidans, e.g., wild type A. ferrooxidans 23270 strain or similar, and the IOB are genetically modified by including at least one of a 2-keto-acid decarboxylase gene (outlined by box) and an alcohol dehydrogenase gene or similar. The production of isobutanol in prokaryotic hosts begins with the amino acid biosynthesis pathways. These pathways produce 2-keto acids, and these are converted to aldehydes using a 2-keto-acid decarboxylase. Alcohol dehydrogenase is then used to convert the aldehydes to alcohols. In the case of isobutanol, the valine biosynthesis pathway is used, and the starting precursor is 2-keto-isovalerate.

In some embodiments, the IOB provided are genetically modified to be able to utilize hydrogen as an electron donor. The use of hydrogen as a mediator improves system efficiency because hydrogen may be cogenerated with ferrous iron during the electrochemical regeneration step. There are various hydrogenase enzymes from different organisms that can be used in microbial biohydrogen production. But other hydrogenase enzymes, found in organisms such as Metallosphaera sedula and Rhodopseudomonas palustris, enable hydrogen uptake and its use as a reductant.

Referring again to FIG. 3, at 204, a first source of ferrous iron is fed to the IOB. At 205, water is fed to the IOB. At 206, carbon dioxide is fed to the IOB. At 207, oxygen is fed to the IOB. At 208, the precipitation of ferric ions is prevented by either adding one or more iron chelators to the bioreactor or its concentration is maintained at low level to avoid precipitation by introduction of a metallic ion such as vanadium with the ferrous iron. The second ion must be able to reduce ferric ions to ferrous, as predicted by a standard table of reduction potentials. Preferably, the oxidized form of the additional metallic ion can be readily reduced by the electrochemical reactor. At 209, a biofuel, ferric iron, and an IOB biomass are produced. In some embodiments, ferric iron production is maximized during 209. In some embodiments, the biofuel is one of isobutanol, a long chain alcohol, or an alkane. At 210, the ferric iron produced is electrochemically reduced to a second source of ferrous iron. Hydrogen is also often produced while electrochemically reducing the ferric iron. Next, at 212, the second source of ferrous iron and the hydrogen are fed to the IOB. The second source of ferrous iron serves as a mediator for transferring electrons to the IOB. Then, the process returns to 209 where additional biofuel, ferric iron, and IOB biomass are produced.

Some embodiments of the disclosed subject matter include methods and systems that do not include the electrochemical regeneration of ferrous iron. For example, where a feed rich in ferrous iron exists, the conversion of ferrous iron and CO₂ to a valuable product (biofuel or other chemical) can be achieved without electrochemical regeneration of ferrous iron. In some cases, e.g., when the chemical product being produced is very valuable, purchased ferrous iron will be used as a feedstock, thus eliminating the need for the electrochemical regeneration of ferrous iron.

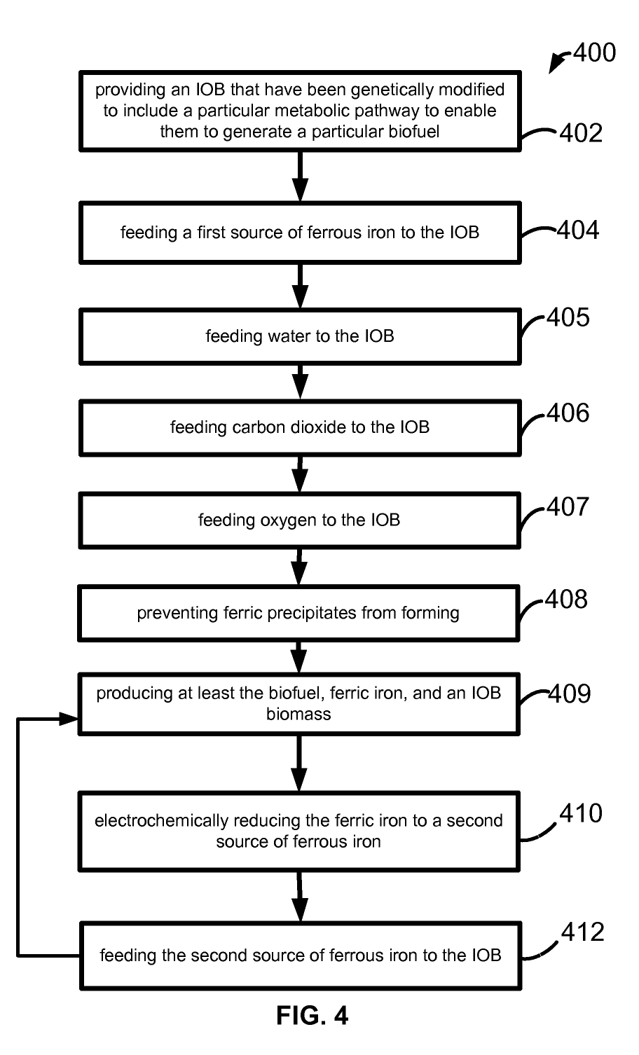

Referring now to FIG. 4, some embodiments include a method 400 for producing a chemical using genetically modified IOB. At 402, IOB that have been genetically modified to include a particular metabolic pathway to enable them to generate a particular chemical are provided. At 404, a first source of ferrous iron is fed to the IOB. At 403, water is fed to the IOB. At 406, carbon dioxide is fed to the IOB. At 407, oxygen is fed to the IOB. At 408, the precipitation of ferric ions is prevented by either adding one or more iron chelators to the bioreactor or mixing a metallic ion such as vanadium with the ferrous iron. At 409, a chemical, ferric iron, and an IOB biomass are produced. At 410, the ferric iron produced is electrochemically reduced to a second source of ferrous iron. Hydrogen is also often produced while electrochemically reducing the ferric iron. Next, at 412, the second source of ferrous iron and the hydrogen are fed to the IOB. Then, the process returns to 409 where additional chemical, ferric iron, and IOB biomass are produced.

Reverse microbial fuel cells according to the disclosed subject matter utilize carbon dioxide and electrical input to produce infrastructure compatible transportation fuels. The technology uses cultures of IOB, e.g., A. ferrooxidans, which are genetically modified to produce isobutanol.

Systems and methods according to the disclosed subject matter use only abundant, inexpensive redox mediators. They do not use costly rare earth elements or organic redox shuttles, and thus can be potentially deployed economically at scale. They potentially exceed an overall efficiency greater than one percent and butanol has desirable fuel properties and is compatible with transportation-fuel infrastructure.

Although the disclosed subject matter has been described and illustrated with respect to embodiments thereof, it should be understood by those skilled in the art that features of the disclosed embodiments can be combined, rearranged, etc., to produce additional embodiments within the scope of the invention, and that various other changes, omissions, and additions may be made therein and thereto, without parting from the spirit and scope of the present invention.

What is claimed is:

1. A method for producing a biofuel using genetically modified iron-oxidizing bacteria (IOB), said method comprising:
providing an IOB that have been genetically modified to enable them to generate a particular biofuel;
feeding a first source of ferrous iron to said IOB;
feeding water, carbon dioxide, and oxygen to said IOB;
producing at least said biofuel, ferric iron, and an IOB biomass; and
preventing ferric precipitates from forming.

2. The method according to claim 1, wherein preventing ferric precipitates from forming includes mixing an iron chelator with said first source of ferrous iron, said IOB, said water, said carbon dioxide, and said oxygen.

3. The method according to claim 1, wherein said iron chelator includes one or more of malonic acid, citric acid, and gluconic acid.

4. The method according to claim 3, wherein said iron chelator includes citric acid at a concentration of about 50 to 300 mM.

5. The method according to claim 1, wherein preventing ferric precipitates from forming includes mixing an indirect electron supplier together with said ferrous iron to form a medium having an increased energy density.

6. The method according to claim 5, wherein said indirect electron supplier includes vanadium.

7. The method according to claim 5, wherein said medium includes about 10-30 mM Fe² mixed with about 50-200 mM V³⁺.

8. The method according to claim 1, further comprising:
electrochemically reducing said ferric iron to a second source of ferrous iron; and
feeding said second source of ferrous iron to said IOB, wherein said second source of ferrous iron serves as a mediator for transferring electrons to said IOB.

9. A system for producing biofuels using genetically modified iron-oxidizing bacteria, said system comprising:
a bioreactor including IOB that have been genetically modified to include a particular metabolic pathway to enable them to generate a particular biofuel;
a first source of ferrous iron in fluid communication with said bioreactor;
a source of water in fluid communication with said bioreactor;
a source of oxygen in fluid communication with said bioreactor;
a source of carbon dioxide in fluid communication with said bioreactor;
a source of anti-ferric precipitating agent in fluid communication with said bioreactor; and
an electrochemical reactor in fluid communication with said bioreactor, said electrochemical reactor configured to electrochemically reduce ferric iron produced in said bioreactor to a second source of ferrous iron.

10. The system according to claim 9, wherein said source of anti-ferric precipitating agent includes an iron chelator.

11. The system according to claim 10, wherein said iron chelator includes one or more of malonic acid, citric acid, and gluconic acid.

12. The system according to claim 11, wherein said iron chelator includes citric acid at a concentration of about 50-300 mM.

13. The system according to claim 9, wherein said source of anti-ferric precipitating agent includes an indirect electron supplier.

14. The system according to claim 13 wherein said indirect electron supplier includes vanadium.

15. The system according to claim 14, wherein said vanadium (V³⁺) has a concentration of about 50-200 mM and said ferrous iron has a concentration of about 10-30 mM.

16. A method for producing a chemical compound using genetically modified iron-oxidizing bacteria, said method comprising:
providing an IOB that have been genetically modified to include a particular metabolic pathway to enable them to generate a particular chemical;
feeding a first source of ferrous iron to said IOB;
feeding water, carbon dioxide, and oxygen to said IOB;
producing at least said chemical compound, ferric iron, and an IOB biomass;
preventing ferric precipitates from forming;
electrochemically reducing said ferric iron to a second source of ferrous iron; and
feeding said second source of ferrous iron to said IOB.

17. The method according to claim 16, wherein preventing ferric precipitates from forming includes mixing an iron chelator with said first source of ferrous iron, said IOB, said water, said carbon dioxide, and said oxygen.

18. The method according to claim 17, wherein said iron chelator includes citric acid at a concentration of about 50-300 mM.

19. The method according to claim 16, wherein preventing ferric precipitates from forming includes mixing an indirect electron supplier together with said ferrous iron to form a medium having an increased energy density.

20. The method according to claim 19, wherein said indirect electron supplier includes vanadium.
